(19) United States
(12) Patent Application Publication
(10) Pub. No.: US 2003/0158500 A1
(43) Pub. Date: Aug. 21, 2003

(54) Decreasing pressure differential viscometer

(76) Inventors: Kenneth Kensey, Malvern, PA (US); William N. Hogenauer, Gilbertsville, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/366,289

(22) Filed: Feb. 13, 2003

Related U.S. Application Data
(63) Continuation-in-part of application No. 09/973,639, filed on Oct. 9, 2001.
Application No. 09/973,639 is a continuation of application No. 09/573,267, filed on May 18, 2000, now Pat. No. 6,402,703.
Application No. 09/573,267 is a continuation-in-part of application No. 09/439,795, filed on Nov. 12, 1999, now Pat. No. 6,322,524.

(51) Int. Cl. A61B 5/00

(52) U.S. Cl. 600/573

(57) ABSTRACT

Apparatus and methods for obtaining the viscosity of a fluid using a continuously decreasing pressure differential that subjects the fluid to a plurality of shear rates and allows data related to that movement to be easily collected and combined with the dimensions of a flow restrictor, through which the fluid passes, to calculate the fluid viscosity.

### RELATED APPLICATIONS

This application is Continuation-in-Part of application Ser. No. 09/973,639, filed on Oct. 9, 2001, which in turn is a Continuation of on application Ser. No. 09/573,267, (now U.S. Pat. No. 6,402,703 filed on May 18, 2000, which in turn is a Continuation-in-Part of application Ser. No. 09/439,795 (now U.S. Pat. No. 6,322,524), filed Nov. 12, 1999 all of which are entitled DUAL RISER/SINGLE CAPILLARY VISCOMETER and all of whose entire disclosures are incorporated by reference herein and both of which are assigned to the same Assignee as the present invention, namely Rheologics, Inc.

### FIELD OF THE INVENTION

The invention pertains to methods and apparatus for determining the viscosity of a fluid, and more particularly, to methods and apparatus for obtaining the viscosity of a fluid using a continuously decreasing pressure differential over plural shear rates.

### BACKGROUND OF INVENTION

The importance of determining the viscosity of blood is well-known. Fibrogen, Viscosity and White Blood Cell Count Are Major Risk Factors for Ischemic Heart Disease, by Yarnell et al., Circulation, Vol. 83, No. 3, March 1991; Postprandial Changes in Plasma and Serum Viscosity and Plasma Lipids and Lipoproteins After an Acute Test Meal, by Tangney, et al., American Journal for Clinical Nutrition, 65:36-40,1997; Studies of Plasma Viscosity in Primary Hyperlipoproteinaemia, by Leonhardt et al., Atherosclerosis 28,29-40, 1977; Effects of Lipoproteins on Plasma Viscosity, by Seplowitz, et al., Atherosclerosis 38, 89-95, 1981; Hyperviscosity Syndrome in a Hypercholesterolemic Patient with Primary Biliary Cirrhosis, Rosenson, et al., Gastroenterology, Vol. 98, No. 5, 1990; Blood Viscosity and Risk of Cardiovascular Events:the Edinburgh Artery Study, by Lowe et al., British Journal of Hematology, 96, 168-171, 1997; Blood Rheology Associated with Cardiovascular Risk Factors and Chronic Cardiovascular Diseases: Results of an Epidemiologic Cross-Sectional Study, by Koenig, et al., Angiology, The Journal of Vascular Diseases, November 1988; Importance of Blood Viscoelasticity in Arteriosclerosis, by Hell, et al., Angiology, The Journal of Vascular Diseases, June, 1989; Thermal Method for Continuous Blood-Velocity Measurements in Large Blood Vessels, and Cardiac-Output Determination, by Delanois, Medical and Biological Engineering, Vol. 11, No. 2, March 1973; Fluid Mechanics in Atherosclerosis, by Nerem, et al., Handbook of Bioengineering, Chapter 21, 1985.

Much effort has been made to develop apparatus and methods for determining the viscosity of blood. Theory and Design of Disposable Clinical Blood Viscometer, by Litt et al., Biorheology, 25, 697-712, 1988; Automated Measurement of Plasma Viscosity by Capillary Viscometer, by Cooke, et al., Journal of Clinical Pathology 41,1213-1216,1988; A Novel Computerized Viscometer/Rheometer by Jimenez and Kostic, Rev. Scientific Instruments 65, Vol 1, January 1994; A New Instrument for the Measurement of Plasma-Viscosity, by John Harkness, The Lancet, pp. 280-281, Aug. 10, 1963; Blood Viscosity and Raynaud's Disease, by Pringle, et al., The Lancet, pp. 1086-1089, May 22, 1965; Measurement of Blood Viscosity Using a Conicylindrical Viscometer, by Walker et al., Medical and Biological Engineering, pp. 551-557, September 1976.

One reference, namely, The Goldman Algorithm Revisited: Prospective Evaluation of a Computer-Derived Algorithm Versus Unaided Physician Judgment in Suspected Acute Myocardial Infarction, by Qamar, et al., Am Heart J 138(4):705-709, 1999, discusses the use of the Goldman algorithm for providing an indicator to acute myocardial infarction. The Goldman algorithm basically utilizes facts from a patient's history, physical examination and admission (emergency room) electrocardiogram to provide an AMI indicator.

In addition, there are a number of patents relating to blood viscosity measuring apparatus and methods. See for example, U.S. Pat. No. 3,342,063 (Smythe et al.); U.S. Pat. No. 3,720,097 (Kron); U.S. Pat. No. 3,999,538 (Philpot, Jr.); U.S. Pat. No. 4,083,363 (Philpot); U.S. Pat. No. 4,149,405 (Ringrose); U.S. Pat. No. 4,165,632 (Weber, et. al.); U.S. Pat. No. 4,517,830 (Gunn, deceased, et. al.); U.S. Pat. No. 4,519,239 (Kiesewetter, et. al.); U.S. Pat. No. 4,554,821 (Kiesewetter, et. al.); U.S. Pat. No. 4,858,127 (Kron, et. al.); U.S. Pat. No. 4,884,577 (Merrill); U.S. Pat. No. 4,947,678 (Hori et al.); U.S. Pat. No. 5,181,415 (Esvan et al.); U.S. Pat. No. 5,257,529 (Taniguchi et al.); U.S. Pat. No. 5,271,398 (Schlain et al.); and U.S. Pat. No. 5,447,440 (Davis, et. al.).

The Smythe '063 patent discloses an apparatus for measuring the viscosity of a blood sample based on the pressure detected in a conduit containing the blood sample. The Kron '097 patent discloses a method and apparatus for determining the blood viscosity using a flowmeter, a pressure source and a pressure transducer. The Philpot '538 patent discloses a method of determining blood viscosity by withdrawing blood from the vein at a constant pressure for a predetermined time period and from the volume of blood withdrawn. The Philpot '363 patent discloses an apparatus for determining blood viscosity using a hollow needle, a means for withdrawing and collecting blood from the vein via the hollow needle, a negative pressure measuring device and a timing device. The Ringrose '405 patent discloses a method for measuring the viscosity of blood by placing a sample of it on a support and directing a beam of light through the sample and then detecting the reflected light while vibrating the support at a given frequency and amplitude. The Weber '632 patent discloses a method and apparatus for determining the fluidity of blood by drawing the blood through a capillary tube measuring cell into a reservoir and then returning the blood back through the tube at a constant flow velocity and with the pressure difference between the ends of the capillary tube being directly related to the blood viscosity. The Gunn '830 patent discloses an apparatus for determining blood viscosity that utilizes a transparent hollow tube, a needle at one end, a plunger at the other end for creating a vacuum to extract a predetermined amount and an apertured weight member that is movable within the tube and is movable by gravity at a rate that is a function of the viscosity of the blood. The Kiesewetter '239 patent discloses an apparatus for determining the flow shear stress of suspensions, principally blood, using a measuring chamber comprised of a passage configuration that simulates the natural microcirculation of capillary passages in a being. The Kiesewetter '821 patent discloses another apparatus for determining the viscosity of fluids, particularly blood, that includes the use of two parallel branches of a flow loop in combination with a flow rate measuring device for measuring the flow in one of the branches for determining the blood viscosity. The Kron '127 patent discloses an apparatus and method for determining blood viscosity of a blood sample over a wide range of shear rates. The Merrill '577 patent discloses an apparatus and method for determining the blood viscosity of a blood sample using a hollow column in fluid communication with a chamber containing a porous bed and means for measuring the blood flow rate within the column. The Hori '678 patent discloses a method for measurement of the viscosity change in blood by disposing a temperature sensor in the blood flow and stimulating the blood so as to cause a viscosity change. The Esvan '415 patent discloses an apparatus that detects the change in viscosity of a blood sample based on the relative slip of a drive element and a driven element, which holds the blood sample, that are rotated. The Taniguchi '529 patent discloses a method and apparatus for determining the viscosity of liquids, e.g., a blood sample, utilizing a pair of vertically-aligned tubes coupled together via fine tubes while using a pressure sensor to measure the change of an internal tube pressure with the passage of time and the change of flow rate of the blood. The Bedingham '328 patent discloses an intravascular blood parameter sensing system that uses a catheter and probe having a plurality of sensors (e.g., an O₂ sensor, CO₂ sensor, etc.) for measuring particular blood parameters in vivo. The Schlain '398 patent discloses a intra-vessel method and apparatus for detecting undesirable wall effect on blood parameter sensors and for moving such sensors to reduce or eliminate the wall effect. The Davis '440 patent discloses an apparatus for conducting a variety of assays that are responsive to a change in the viscosity of a sample fluid, e.g., blood.

Viscosity measuring methods and devices for fluids in general are well-known. See for example, U.S. Pat. No. 1,810,992 (Dallwitz-Wegner); U.S. Pat. No. 2,343,061 (Irany); U.S. Pat. No. 2,696,734 (Brunstrum et al.); U.S. Pat. No. 2,700,891 (Shafer); U.S. Pat. No. 2,934,944 (Eolkin); U.S. Pat. No. 3,071,961 (Heigl et al.); U.S. Pat. No. 3,116,630 (Piros); U.S. Pat. No. 3,137,161 (Lewis et al.); U.S. Pat. No. 3,138,950 (Welty et al.); U.S. Pat. No. 3,277,694 (Cannon et al.); U.S. Pat. No. 3,286,511 (Harkness); U.S. Pat. No. 3,435,665 (Tzentis); U.S. Pat. No. 3,520,179 (Reed); U.S. Pat. No. 3,604,247 (Gramain et al.); U.S. Pat. No. 3,666,999 (Moreland, Jr. et al.); U.S. Pat. No. 3,680,362 (Geerdes et al.); U.S. Pat. No. 3,699,804 (Gassmann et al.); U.S. Pat. No. 3,713,328 (Aritomi); U.S. Pat. No. 3,782,173 (Van Vessem et al.); U.S. Pat. No. 3,864,962 (Stark et al.); U.S. Pat. No. 3,908,441 (Virloget); U.S. Pat. No. 3,952,577 (Hayes et al.); U.S. Pat. No. 3,990,295 (Renovanz et al.); U.S. Pat. No. 4,149,405 (Ringrose); U.S. Pat. No. 4,302,965 (Johnson et al.); U.S. Pat. No. 4,426,878 (Price et al.); U.S. Pat. No. 4,432,761 (Dawe); U.S. Pat. No. 4,616,503 (Plungis et al.); U.S. Pat. No. 4,637,250 (Irvine, Jr. et al.); U.S. Pat. No. 4,680,957 (Dodd); U.S. Pat. No. 4,680,958 (Ruelle et al.); U.S. Pat. No. 4,750,351 (Ball); U.S. Pat. No. 4,856,322 (Langrick et al.); U.S. Pat. No. 4,899,575 (Chu et al.); U.S. Pat. No. 5,142,899 (Park et al.); U.S. Pat. No. 5,222,497 (Ono); U.S. Pat. No. 5,224,375 (You et al.); U.S. Pat. No. 5,257,529 (Taniguchi et al.); U.S. Pat. No. 5,327,778 (Park); and U.S. Pat. No. 5,365,776 (Lehmann et al.).

The following U.S. patents disclose viscosity or flow measuring devices, or liquid level detecting devices using optical monitoring: U.S. Pat. No. 3,908,441 (Virloget); U.S. Pat. No. 5,099,698 (Kath, et. al.); U.S. Pat. No. 5,333,497 (Br nd Dag A. et al.). The Virloget '441 patent discloses a device for use in viscometer that detects the level of a liquid in a transparent tube using photodetection. The Kath '698 patent discloses an apparatus for optically scanning a rotameter flow gauge and determining the position of a float therein. The Br nd Dag A. '497 patent discloses a method and apparatus for continuous measurement of liquid flow velocity of two risers by a charge coupled device (CCD) sensor.

U.S. Pat. No. 5,421,328 (Bedingham) discloses an intravascular blood parameter sensing system.

A statutory invention registration, H93 (Matta et al.) discloses an apparatus and method for measuring elongational viscosity of a test fluid using a movie or video camera to monitor a drop of the fluid under test.

The following publications discuss red blood cell deformability and/or devices used for determining such: Measurement of Human Red Blood Cell Deformability Using a Single Micropore on a Thin Si₃N₄ Film, by Ogura et al, IEEE Transactions on Biomedical Engineering, Vol. 38, No. 8, August 1991; the Pall BPF4 High Efficiency Leukocyte Removal Blood Processing Filter System, Pall Biomedical Products Corporation, 1993.

A device called the “Hevimet 40” has recently been advertised at www.hevimet.freeserve.co.uk. The Hevimet 40 device is stated to be a whole blood and plasma viscometer that tracks the meniscus of a blood sample that falls due to gravity through a capillary. While the Hevimet 40 device may be generally suitable for some whole blood or blood plasma viscosity determinations, it appears to exhibit several significant drawbacks. For example, among other things, the Hevimet 40 device appears to require the use of anti-coagulants. Moreover, this device relies on the assumption that the circulatory characteristics of the blood sample are for a period of 3 hours the same as that for the patient's circulating blood. That assumption may not be completely valid.

Thus, there remains a need for determining the viscosity of a fluid over a plurality of shear rates without the need to detect very small pressure differentials, especially where the fluid is blood and without the need to adulterate the blood, thereby permitting a more accurate and quick method for determining the blood viscosity.

### SUMMARY OF THE INVENTION

An apparatus for determining the viscosity of a fluid over plural shear rates using a continuously decreasing pressure differential. The apparatus comprises: a lumen (e.g., a riser tube) for supporting a column of fluid therein and wherein the column of fluid has a start point defined above a horizontal reference position. The lumen comprises: a first end and a second end, a flow restrictor (e.g., a capillary tube) for restricting the movement of the column of fluid and located between the first and second ends and wherein the flow restrictor comprises some known dimensions (e.g., diameter and length); and wherein, after the start point has been defined, the first and second ends are exposed to atmospheric pressure to subject the column of fluid to a continuously decreasing pressure differential that causes the column of fluid to move away from the start point towards the second end through a plurality of shear rates; a sensor (an optical detector, a mass detector, time of flight detector, etc.) for monitoring the movement of the column of fluid in the lumen for generating data related to the movement (e.g., changing column height, changing mass, etc.); and a processor for using the data and said some known dimensions to calculate the viscosity of the fluid.

A method for determining the viscosity of a fluid over plural shear rates using a continuously decreasing pressure differential. The method comprises the steps of: forming a column of the fluid in a substantially upright lumen (e.g., a riser tube) having a first end and a second end; establishing a start point of the column of fluid above a horizontal reference position; exposing the first end and the second end to atmospheric pressure to subject the column of fluid to a continuously decreasing pressure differential that causes the column of fluid to move away from the start point towards the second end through a plurality of shear rates; restricting the movement of the column of fluid by passing at least a portion of the column of fluid through a flow restrictor (e.g., a capillary tube) having some known dimensions (e.g., diameter and length); monitoring the movement of the column of fluid through the plurality of shear rates to generate data related to the movement (e.g., changing column height, changing mass, etc.); and calculating the viscosity of the fluid using the data and the some known dimensions.

A method for determining the viscosity of a fluid over plural shear rates using a continuously decreasing pressure differential. The method comprises the steps of: forming a column of the fluid in a substantially upright lumen (e.g., a capillary tube) having a first end and a second end and wherein the lumen has some known dimensions (e.g., diameter and length); establishing a start point of the column of fluid above a horizontal reference position; exposing the first end and said second end to atmospheric pressure to subject the column of fluid to a continuously decreasing pressure differential that causes the column of fluid to move away from the start point towards the second end through a plurality of shear rates and wherein the substantially upright lumen restricts the movement of the column of fluid as it moves; monitoring the movement of the column of fluid through the plurality of shear rates to generate data related to the movement (e.g., changing column height, changing mass, etc.); and calculating the viscosity of the fluid using the data and the some known dimensions.

### DESCRIPTION OF THE DRAWINGS

FIG. 1 is a flowchart of the method of the present invention based on a continuously decreasing pressure differential (DPD) viscometer;

FIG. 2A is a functional diagram of a fluid under test using a first DPD viscometer, a dual riser/single capillary (DRSC) viscometer, as discussed in U.S. Pat. Nos. 6,322,524 or 6,402,703;

FIG. 2B is a graphical representation of the height of the respective columns of fluid over time in the two riser tubes of the DRSC viscometer as discussed in U.S. Pat. Nos. 6,322,524 and 6,402,703;

FIG. 2C is a functional diagram of the DRSC viscometer as discussed in U.S. Pat. Nos. 6,322,524 and 6,402,703 wherein the circulating blood of a living being is the fluid under test;

FIG. 2D is a front view of an embodiment of the DRSC viscometer of FIG. 2C;

FIG. 2E is an alternative functional diagram of the DRSC viscometer as discussed in U.S. Pat. Nos. 6,322,524 or 6,402,703 wherein the circulating blood of a living being is the fluid under test;

FIG. 2F is a front view of an embodiment of the DRSC viscometer of FIG. 2E;

FIG. 3A is a functional diagram of a second DPD viscometer, a single riser/single capillary tube viscometer using mass detection, showing the column of fluid at a starting point;

FIG. 3B is a functional diagram of the single riser/single capillary tube viscometer using mass detection of FIG. 3A, showing the column of fluid at the end of the viscosity test run;

FIG. 3C is a functional diagram of the single riser/single capillary tube viscometer of FIGS. 3A-3B but using column height detection;

FIG. 3D is a graphical representation of the changing mass over time of the fluid collector from the riser tube of the SRSC viscometers as discussed in U.S. Pat. Nos. 6,412,336 and 6,484,565 corresponding to FIGS. 3A-3B;

FIG. 3E is a graphical representation of the height of the column of fluid over time in the riser tube of the SRSC viscometers as discussed in U.S. Pat. Nos. 6,412,336 and 6,484,565;

FIG. 4A is a functional diagram of a variation of the second DPD viscometer, a single riser/single capillary tube blood viscometer using mass detection;

FIG. 4B is a functional diagram of a variation of the single riser/single capillary tube blood viscometer using column height detection;

FIG. 5A is a functional diagram of a third DPD viscometer, a single capillary tube viscometer;

FIG. 5B is an embodiment of the single capillary tube viscometer of FIG. 5A used for blood viscosity determinations.

### DETAILED DESCRIPTION OF THE PREFERRED EMBODIMENTS

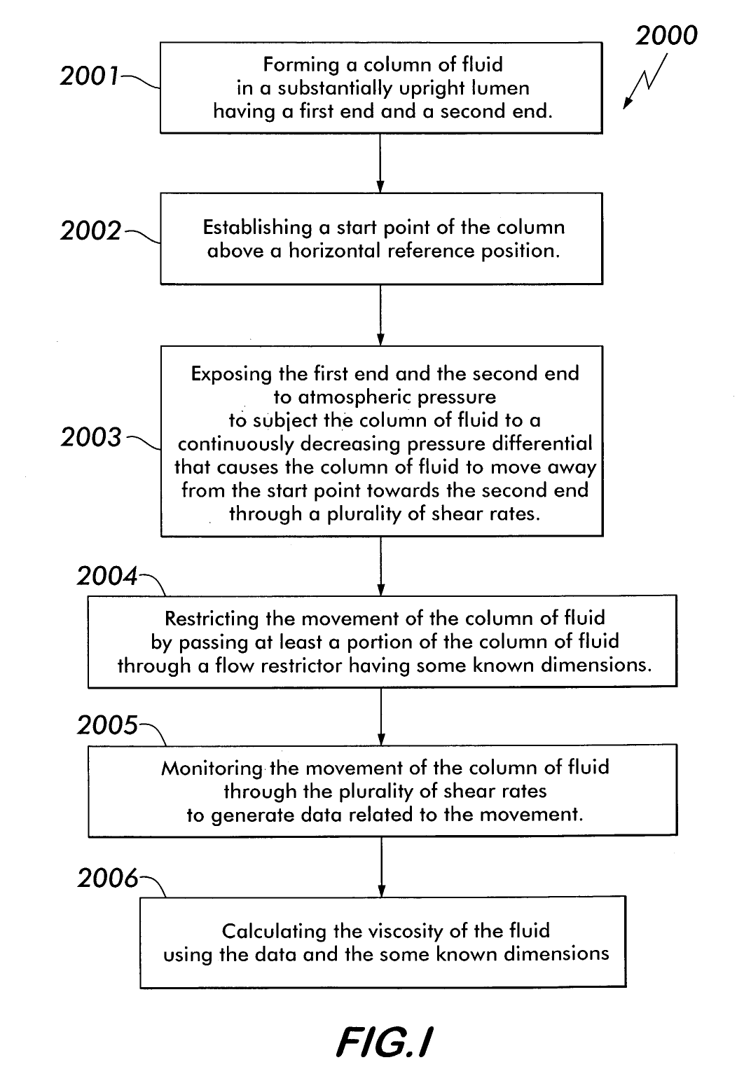

The method 2000 (FIG. 1) of the present invention involves utilizing “decreasing pressure differential (DPD) viscometers” which are owned by Rheologics, Inc. of Exton, Pa. Examples of DPD viscometers are the subject matter of the following U.S. patents and applications, all of which are assigned to the same Assignee, namely Rheologics, Inc., as the present application, and all of whose entire disclosures are incorporated by reference herein:

| U.S. Pat. No. or | |
|---|---|
| Application Serial No. | Title |
| 6,322,524 | Dual Riser/Single Capillary Viscometer |
| 6,402,703 | Dual Riser/Single Capillary Viscometer |
| 6,412,336 | Single Riser/Single Capillary Blood |
| | Viscometer Using Mass Detection or Column |
| | Height Detection |
| 6,450,974 | Method of Isolating Surface Tension and |
| | Yield Stress in Viscosity Measurements |
| 6,484,565 | Single Riser/Single Capillary Viscometer |
| | Using Mass Detection or Column Height |
| | Detection |
| 09/908,374 | Single Capillary Tube Viscometer |
| 10/245,237 | Method for Determining a Characteristic |
| | Viscosity-Shear Rate Relationship for a Fluid |

As referred to throughout this Specification, the viscometers can be used to determine the viscosity of non-biological fluid as well as biological fluids (e.g., blood, plasma, etc.). Once a column of the fluid under test is formed in the viscometer, the fluid is subjected to a plurality of shear rates using a decreasing pressure differential. The device monitors or detects the laminar movement of the fluid as it passes through the plurality of shear rates and then from this laminar movement, as well as using known dimensions of the passageways in the viscometer, the viscosity of the fluid can be accurately and quickly determined. Where biological fluids are concerned, e.g., blood, the viscometers are configured to operate by immediately diverting a portion of the living being's blood into the viscometer which then subjects the blood to a plurality of shear rates using the decreasing pressure differential. The device monitors or detects the laminar movement of the blood as it passes through the plurality of shear rates and then from this laminar movement, as well as using known dimensions of the passageways in the viscometer, the viscosity of the circulating blood can be accurately and quickly determined. The diverted blood remains unadulterated throughout the analysis. Thus, where non-biological fluids are concerned, the viscometer does not have to operate with such expediency but the subjection of the non-biological fluid to the continuously decreasing pressure differential is similar to that of the biological fluid.

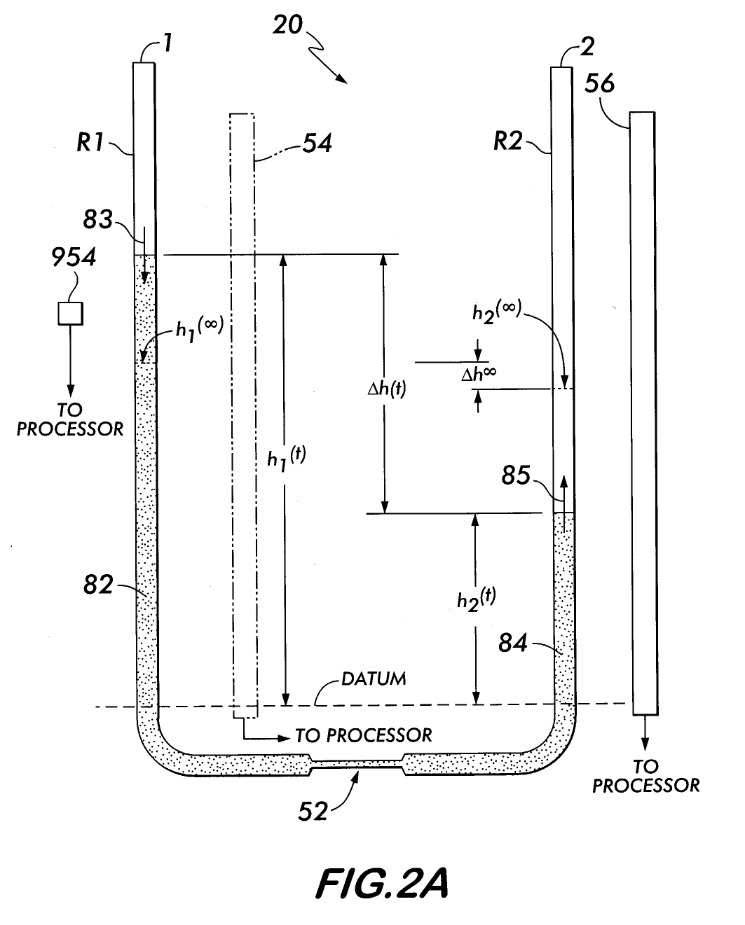

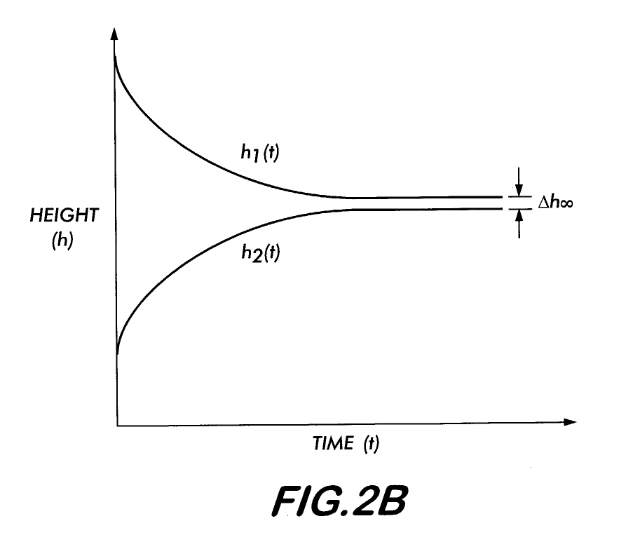

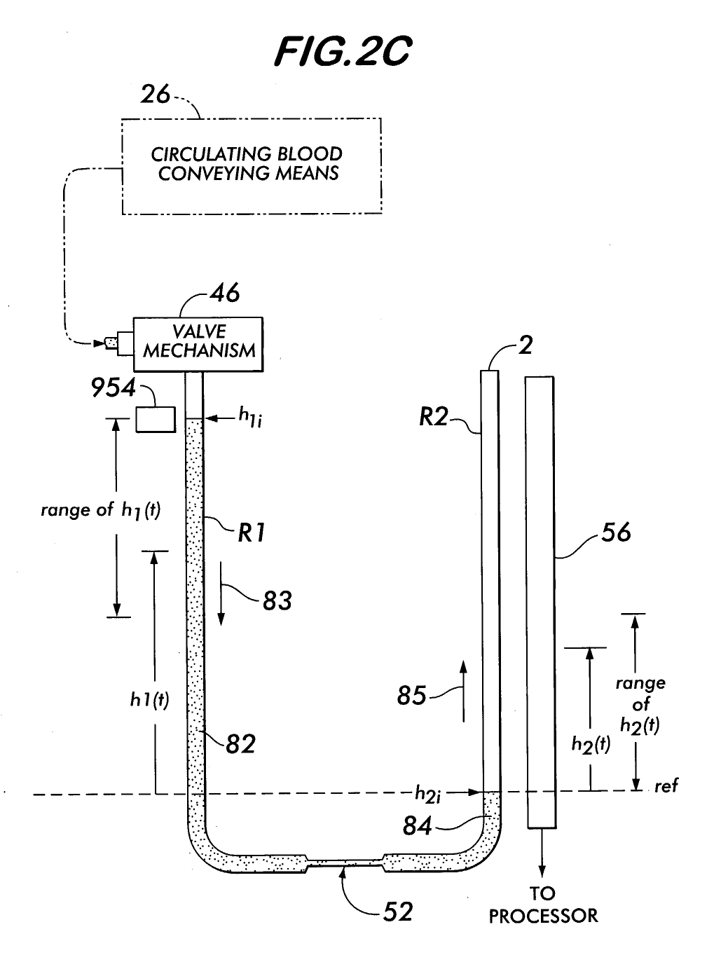

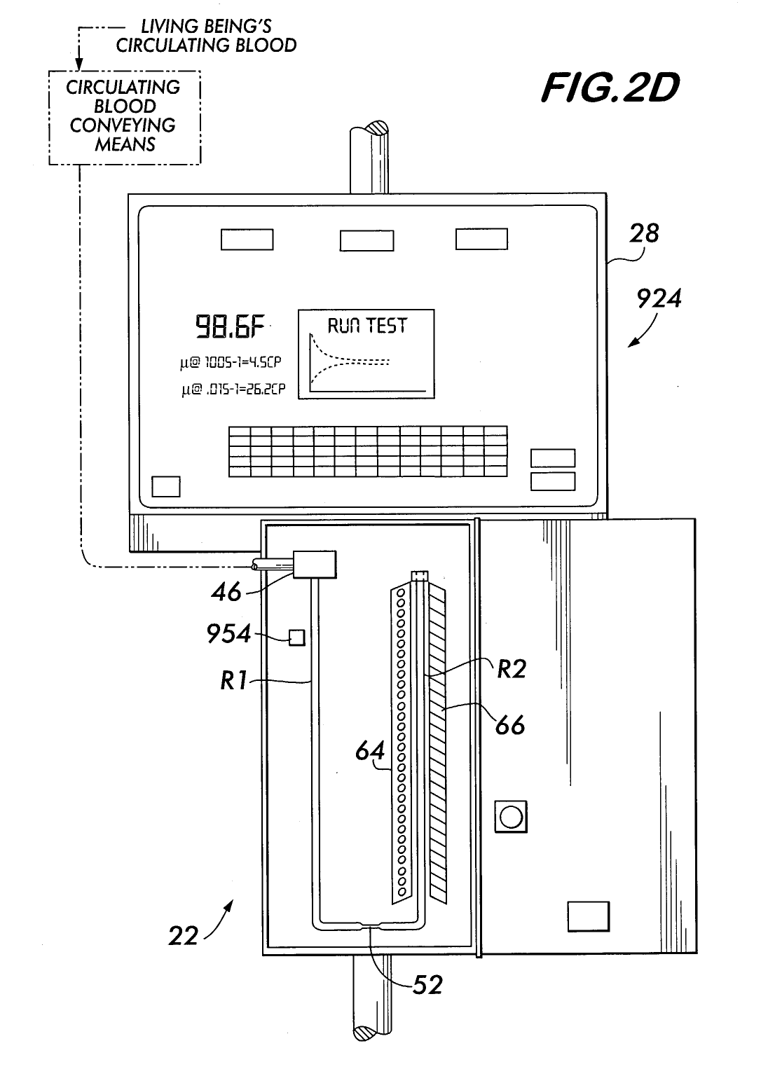

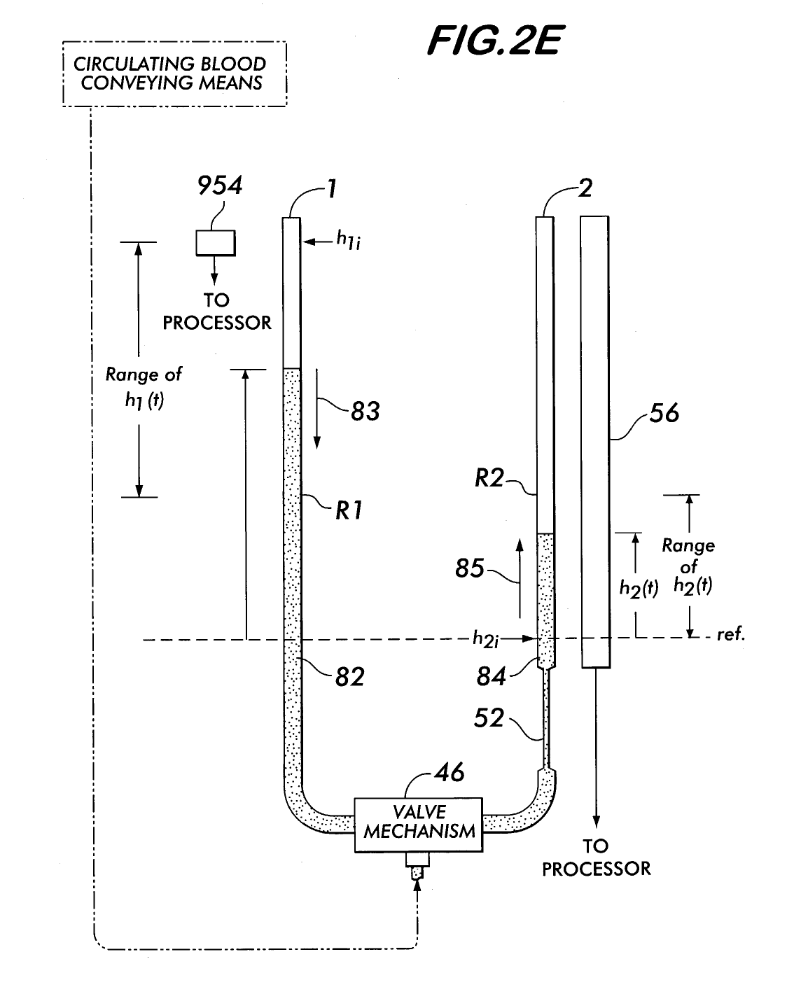

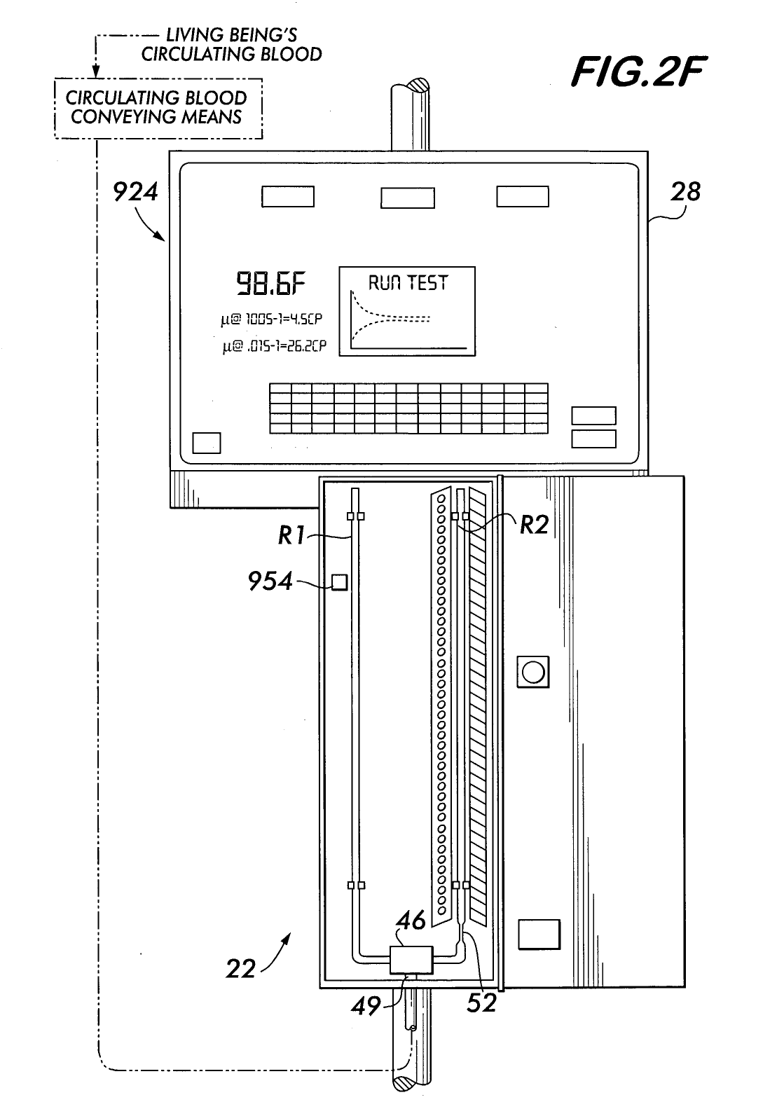

One example of a DPD viscometer is the dual riser/single capillary (DRSC) viscometer 20 of U.S. Pat. Nos. 6,322,524 and 6,402,703 which, when used with a sensor and processor, determines the viscosity of a fluid (e.g., the circulating blood of a living being) over plural shear rates. FIGS. 2A-2F pertain to the inventions of U.S. Pat. Nos. 6,322,524 and 6,402,703.

The DRSC viscometer basically comprises a lumen in the form of a U-shaped structure wherein a portion of that U-shaped structure comprises a flow restrictor, e.g., a capillary tube. The DRSC viscometer is arranged to establish two oppositely moving columns of blood which experience a decreasing pressure differential. The movement of at least one of the columns of blood is detected over time (e.g., using a column level detector, a mass detector, etc.). From this data and using the dimensions of the flow restrictor, the viscosity can be determined (see U.S. Pat. Nos. 6,322,524 and/or 6,402,703).

FIG. 2A depicts the concept of the DRSC viscometer 20 wherein the U-shaped structure comprises a pair of riser tubes, R1 and R2, and a flow restrictor 52. The movement of the columns of blood 82 and 84 in the respective directions 83 and 85 are monitored by respective column level detectors 54 and 56 (U.S. Pat. No. 6,322,524); or alternatively, one of the column level detectors, e.g., 54, can be replaced by a single point detector 954 (U.S. Pat. No. 6,402,703). After a starting point is established (e.g, h₁ᵢ and/or h₂ᵢ, FIG. 2C), the ends (1 and 2) of R1 and R2 are exposed to atmospheric pressure, whereby a decreasing pressure differential, ρgh(t) (where ρ is the density of the fluid under test, g is the gravitational constant, and h(t) is the changing column of fluid height, h₁(t) and/or h₂(t)) causes the fluid column 82 (FIG. 2A) to fall and the fluid column 84 (FIG. 2A) to rise at continuously decreasing shear rates. The sensors generate height data, h₁(t) and h₂(t), over time and provide this data to a computer (not shown). At the end of the viscosity test run, the height of the two columns, namely h₁(∞) and h₂(∞), are not equal and the result is a Δh_{∞} the cause of which may be attributed to surface tension and yield stress of the fluid. FIG. 2B depicts a height vs. time plot for each of the columns of fluid. The processor calculates the fluid viscosity from the height data and the dimensions of the flow restrictor 52. The details of the how the fluid viscosity is calculated using the DRSC viscometer 20 is set forth in U.S. Pat. Nos. 6,322,524 and 6,402,703, both of whose entire disclosures are incorporated by reference herein and as a result will not be discussed further.

As also discussed in U.S. Pat. Nos. 6,322,524 and 6,402,703, where the fluid under test is a biological fluid, (e.g., the circulating blood of a living being), in order to rapidly generate the oppositely-moving columns of blood from the diverted circulating blood of the living being, a valve mechanism 46 is also utilized with the DRSC viscometer and is controlled by the computer. Depending on where the flow restrictor 52 is positioned in the U-shaped structure, the valve mechanism 46 position is selected. For example, in FIG. 2C, the concept of the DRSC viscometer using a flow restrictor 52 at the base of the U-shaped structure has the valve mechanism 46 positioned at the top of the riser tube R1. An embodiment of the DRSC viscometer of FIG. 2C is depicted in FIG. 2D; the embodiment basically comprises a blood receiving means 22 that houses the U-shaped structure and an analyzer 924 portion that includes the processor and a display screen 28 for providing the operator with viscosity, and other critical, data. An alternative configuration is shown in FIGS. 2E and 2F. FIG. 2E shows the concept of the DRSC viscometer using a flow restrictor 52 as part of one of the riser tubes, e.g., R2, and FIG. 2F is an embodiment of that concept.

It should also be understood that the entire disclosure of U.S. Pat. No. 6,450,974 entitled A METHOD OF ISOLATING SURFACE TENSION & YIELD STRESS IN VISCOSITY MEASUREMENTS, which is assigned to the same Assignee as the present invention, namely, Rheologics, Inc., is incorporated by reference herein with regard to the DRSC viscometer 20. In that patent, a methodology is disclosed in which the surface tension and yield stress effects of the fluid under test are isolated from the viscosity measurements. Furthermore, it should be understood that the entire disclosure of application Ser. No. 10/245,237, filed on Sep. 17, 2002 entitled METHOD FOR DETERMINING A CHARACTERISTIC VISCOSITY-SHEAR RATE RELATIONSHIP FOR A FLUID, which is which is assigned to the same Assignee as the present invention, namely, Rheologics, Inc., is incorporated by reference herein with regard to the DRSC viscometer 20. In this application, a methodology is disclosed for generating a characteristic viscosity-shear rate relationship for a fluid, using a DPD viscometer, preferably using a DRSC viscometer.

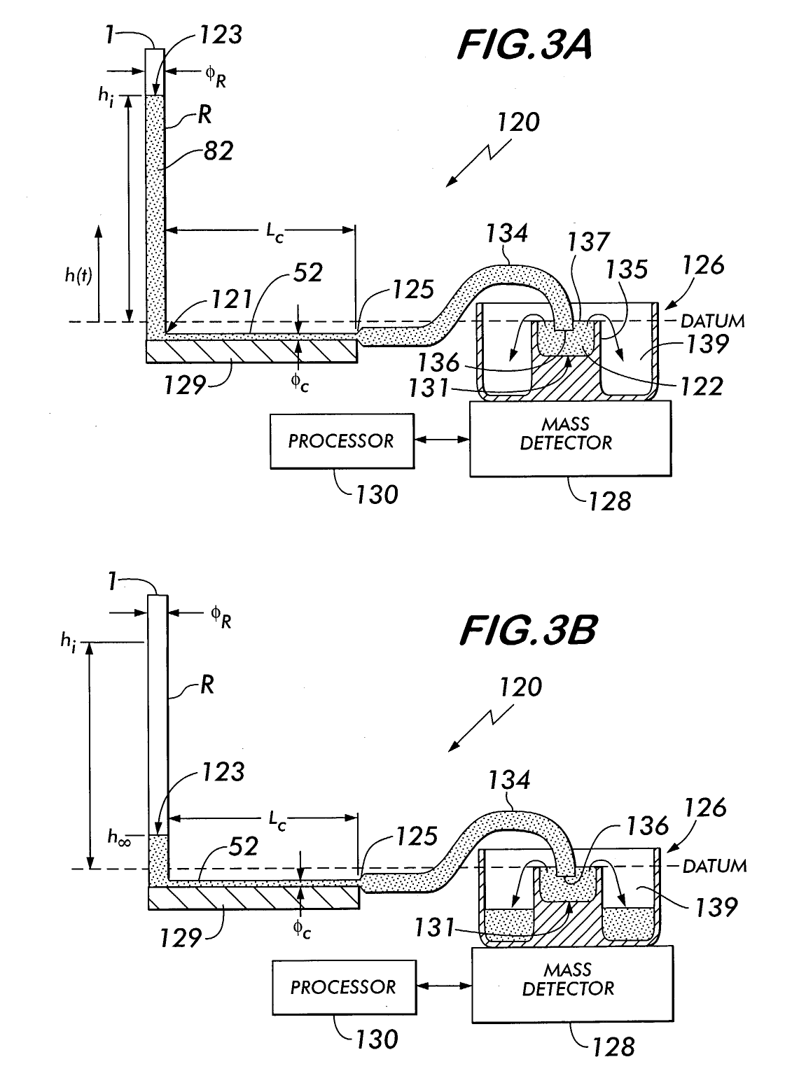

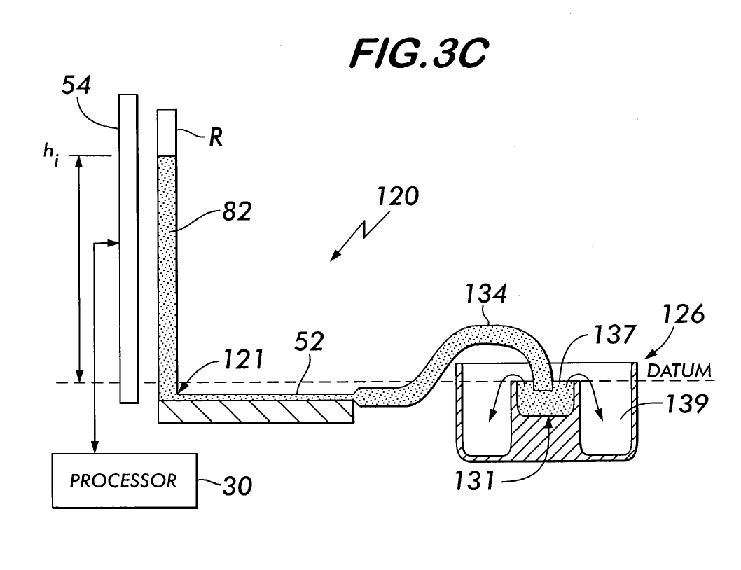

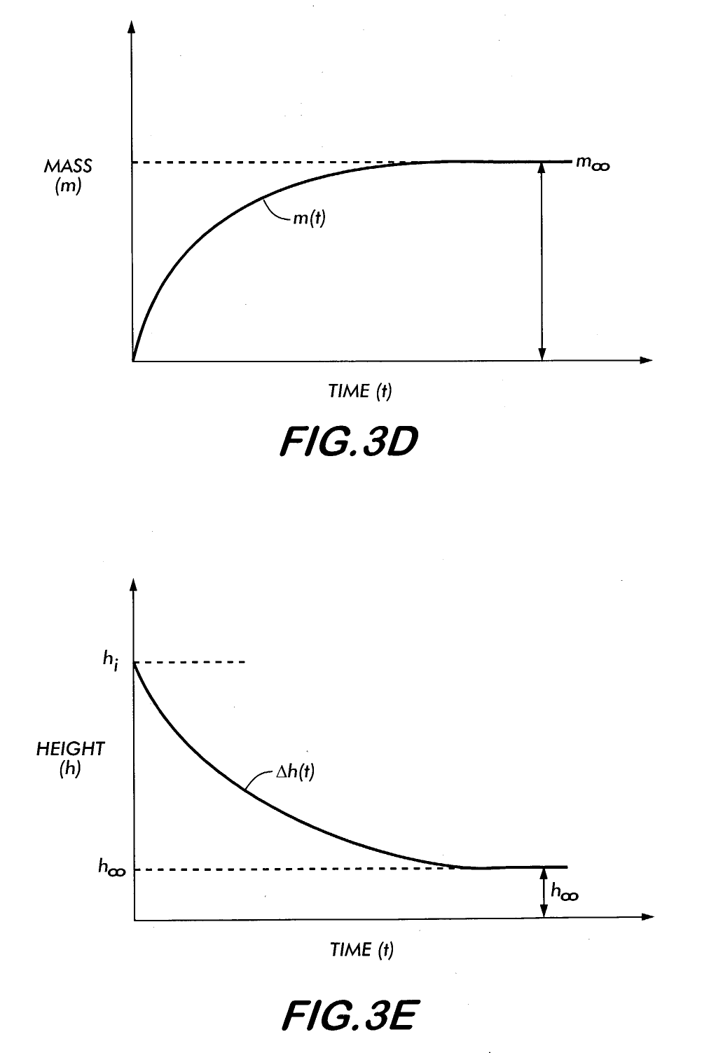

A second example of a DPD viscometer is shown in FIGS. 3A-3C and is known as a single riser/single capillary (SRSC) viscometer using mass detection or column height detection and which forms the subject matter of U.S. Pat. No. 6,484,565 entitled SINGLE RISER/SINGLE CAPILLARY VISCOMETER USING MASS DETECTION OR COLUMN HEIGHT DETECTION, and whose entire disclosure is incorporated by reference herein. This SRSC viscometer 120 utilizes a falling column of fluid under the influence of a decreasing pressure differential to detect either the changing mass (FIGS. 3A-3B), or the changing height (FIG. 3C), of the column of fluid in a lumen as the column moves through a plurality of shear rates. The lumen comprises an “L-shaped” structure, e.g., a single riser tube R having a flow restrictor 124 and adapter 134. The SRSC viscometer 120 utilizes a specialized fluid collector 126 which maintains an output end 136 (which corresponds to the second end 2 of the DRSC viscometer 20) of the adaptor 134 submerged in the fluid that is collecting in the fluid collector 126; this minimizes any surface tension effects that would normally occur if the output 136 of the flow restrictor 124 were simply positioned over the collector 126. In operation, when the first end 1 (FIG. 3A) and the output end 136 are exposed to atmospheric pressure, the column of fluid 138 falls, from a starting point, hᵢ, through a plurality of shear rates under the influence of the decreasing pressure differential which is detected either by a mass detector 128 (FIGS. 3A-3B) or the column level detector 154 (FIG. 3C). FIG. 3D graphically depicts the increasing mass of the fluid collector as the fluid passes out of the lumen into the fluid collector 126 where the mass detector 130 is used; FIG. 3E depicts a height vs. time plot for the column of fluid 82 where column level detector 154 is used. The processor calculates the fluid viscosity from the height data and the dimensions of the flow restrictor 52 In accordance with the disclosure set forth in the U.S. Pat. No. 6,484,565, the fluid viscosity is then determined from this detected data along with dimensions of the passageways in the device 120.

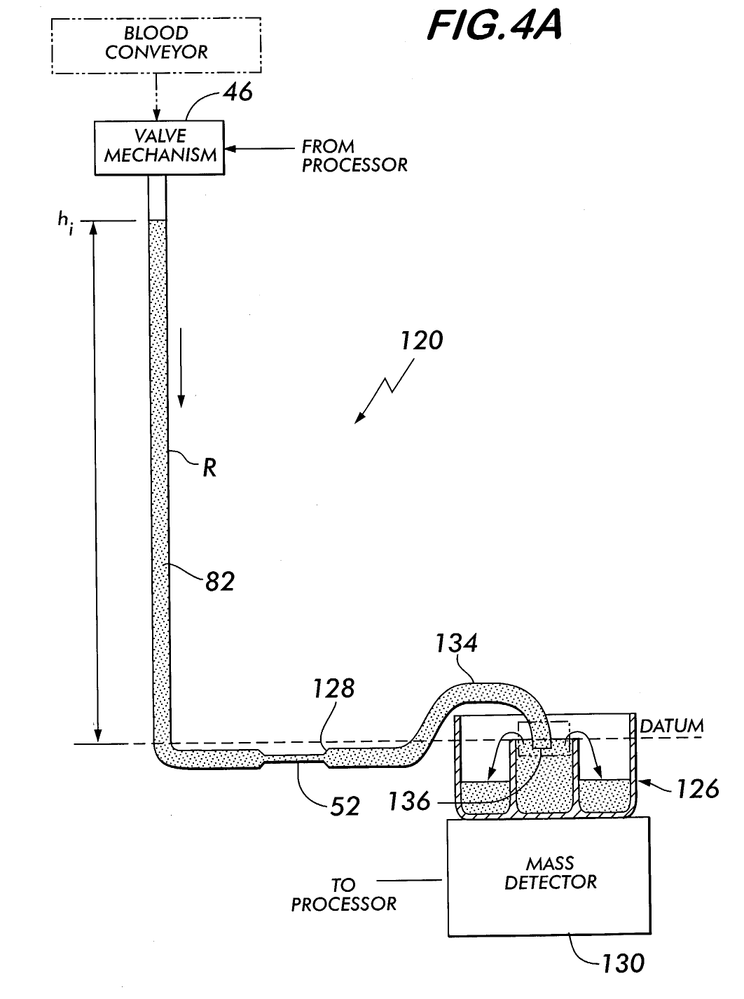

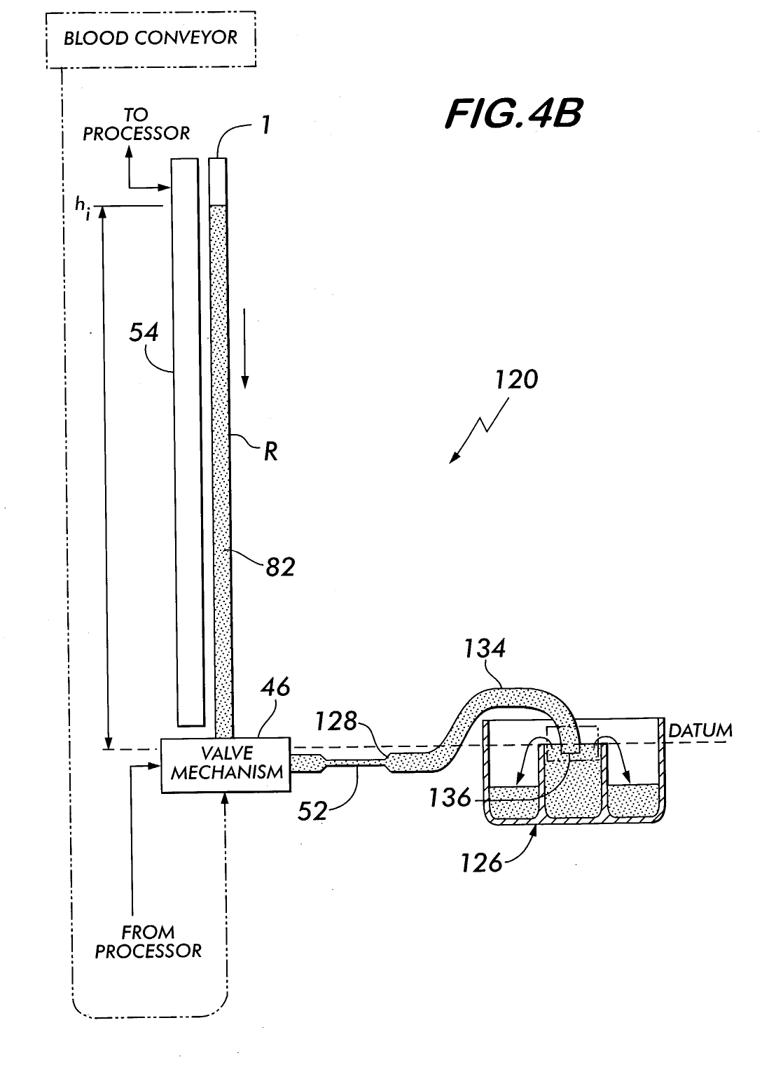

A specialized use of the SRSC viscometer is shown in U.S. Pat. No. 6,412,336 in which the fluid under test is the circulating blood of a living being. In particular, the SRSC blood viscometer 120 utilizes a falling column of blood under the influence of a decreasing pressure differential to detect either the changing mass of the column of blood 82 in a single riser tube R (FIG. 4A) or the changing height of the column of blood 82 (FIG. 4B) as the column moves through a plurality of shear rates. The SRSC blood viscometer is 120 utilizes the specialized blood collector 126 which maintains an output end 124 of an adaptor 134 submerged in blood that is collecting in the blood collector 126; this minimizes any surface tension effects that would normally occur if the output 124 of the flow restrictor 52 were simply positioned over the collector 126. In operation, the column of blood 82 falls through a plurality of shear rates under the influence of the decreasing pressure differential which is detected either by a mass detector 130 or the column level detector 54. As with the DRSC viscometer 20, the SRSC viscometer 120 utilizes a valve mechanism 46 to rapidly generate the column of blood that is diverted from the living being's circulating blood; and, depending upon where the flow restrictor 52 is positioned in the L-shaped structure, the valve mechanism 46 is located. In accordance with the disclosure set forth in the U.S. Pat. No. 6,412,336, the circulating blood viscosity is then determined from this detected data along with dimensions of the passageways in the device 120.

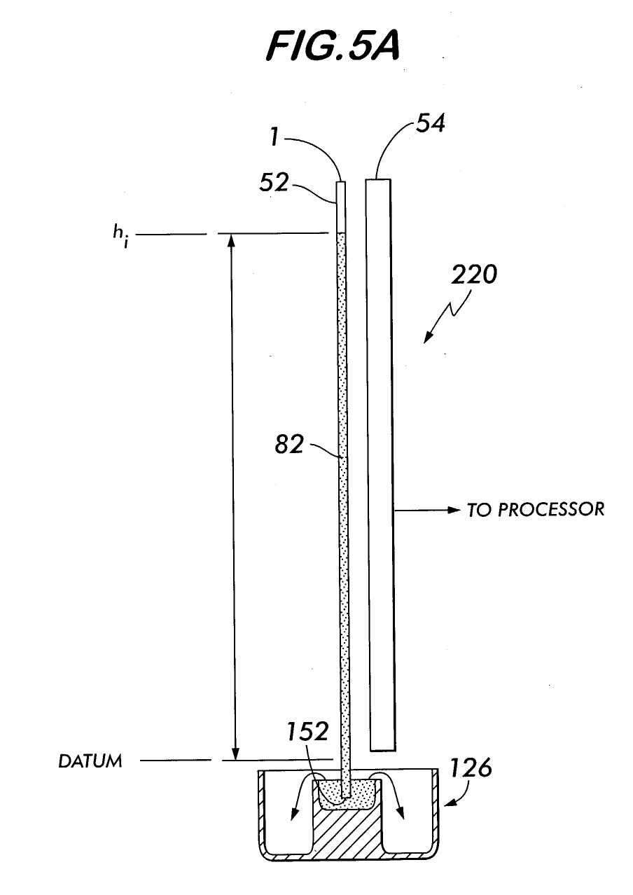

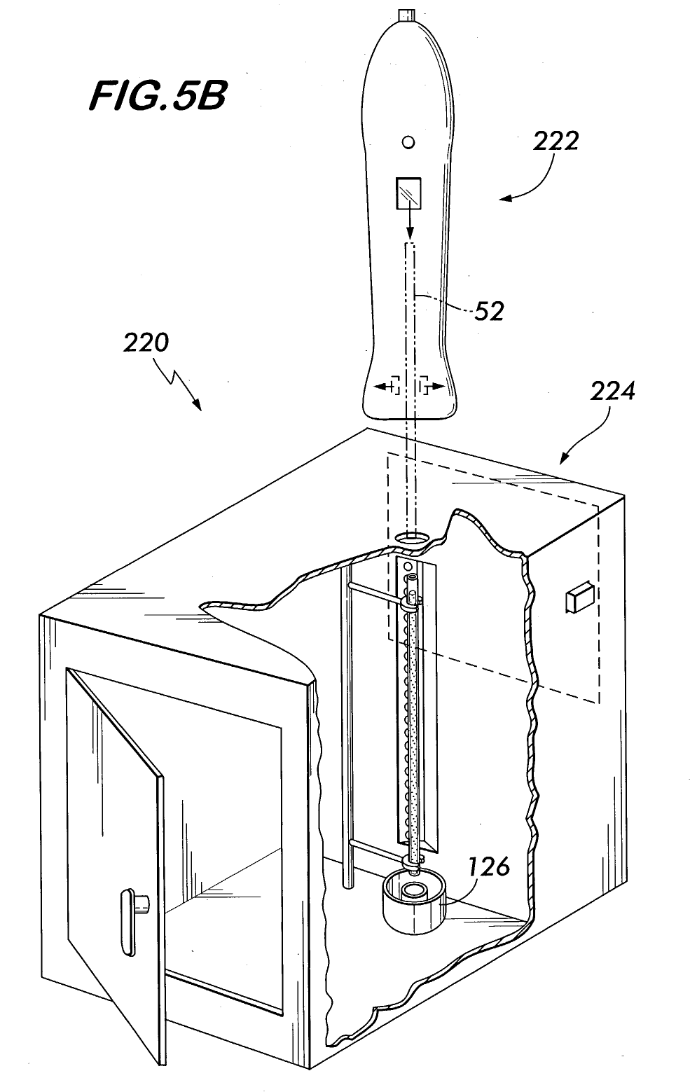

A third example of a DPD viscometer is shown in FIGS. 5A-5B and is known as a single capillary tube viscometer (SCTV) which forms the subject matter of application Ser. No. 09/908,374 filed on Jul. 18, 2001 entitled “SINGLE CAPILLARY TUBE VISCOMETER”, and whose entire disclosure is incorporated by reference herein. This SCTV 220 also utilizes a falling column of fluid 82 under the influence of a decreasing pressure differential to detect the changing height of the column of fluid 82 as the column moves through a plurality of shear rates. However, this device uses only a capillary tube 52 whose output end 152 (which corresponds to the second end 2 of the DRSC viscometer 20) is also submerged in fluid collecting in the collector 126 to minimize surface tension effects. In operation, when the first end 1 (FIG. 5A) and the output end 152 are exposed to atmospheric pressure, the column of fluid 82 falls, from a starting point, hᵢ, through a plurality of shear rates under the influence of the decreasing pressure differential which is detected either by a mass detector 128 (FIGS. 3A-3B) or the column level detector 154 (FIG. 3C).

FIG. 5B depicts an exemplary embodiment of the SCTV 220 where the fluid under test is the circulating blood of a living being. In particular, and in accordance with the application Ser. No. 09/908,374, the SCTV 120 comprises a hand-held portion 222 and an analyzer portion 224. The hand-held portion 222 initially contains the capillary tube 52 and permits blood to be withdrawn from the living being and into the capillary tube 52. The hand-held portion 222 is then immediately interfaced with the analyzer portion 224 and the filled capillary tube 52 is released into the analyzer portion 224. With the filled capillary tube 52 inserted into the analyzer portion 224, the SCTV 220 is formed (as shown in FIG. 5A) and the blood viscosity analysis begins immediately.

It is within the broadest scope of the invention to include any means and/or method for detecting the movement of the columns of fluid in the riser tubes R1, R2, R or capillary tube 52 and, as such, is not limited to the LED array 64/CCD 66 (FIG. 2D) arrangement (U.S. Pat. Nos. 6,322,524 and 6,402,703) nor even limited to the column level detectors 54/56. In fact, the following type of physical detections are covered by the present invention:

d(Weight)/dt: the change in weight of each column of fluid with respect to time using a weight detecting means for each column of fluid as the sensor; e.g., w₁ (t)-w₂ (t);

d(Pressure)/dt: the change in pressure of each column of fluid with respect to time using a pressure transducer located at the top of each column of fluid; e.g., p₁ (t)-p₂ (t);

time of flight: the length of time it takes an acoustic signal to be emitted from a sensor (e.g., ultrasonic) located above each column of fluid and to be reflected and return to the sensor; e.g., time of flight₁(t)-time of flight₂(t);

d(Volume)/dt: the change in volume of each column of fluid with respect to time; e.g., V₁(t)-V₂(t);

d(Position)/dt: the change in position of each column level using a digital video camera; e.g., Pos₁ (t)-Pos₂ (t);

d(Mass)/dt: the change in mass with respect to time for each column of fluid; e.g., m₁ (t)-m₂ (t).

Thus, it should be understood that the manner in which the movement of the column, or columns, of fluid are monitored/detected does not in any way limit the scope of the present invention. The key feature is that the movement of the fluid, caused by a continuously decreasing pressure differential which subjects the fluid to a plurality of shear rates, is monitored or detected and corresponding data is generated related to that movement.

As stated in U.S. Pat. Nos. 6,322,524 and 6,402,703, there are a plurality of mathematical models that can be used as curve fitting models for the data obtained from the DRSC viscometers, such as a power law model, a Casson model (e.g., see application Ser. No. 10/245,237), a Carreau model, a Herschel-Bulkley model, a Powell-Eyring model, a Cross model, Carreau-Yasuda model and it is within the broadest scope of those inventions, as well as the present invention, to include all of these models. And although a power law model was used in those disclosures, that model was used by way of example only. Similarly, a plurality of mathematical models can be used as curve fitting models for the data obtained using the SRSC viscometers, as disclosed in U.S. Pat. Nos. 6,412,336 and 6,484,565 and thus the models used in those disclosures are by way of example only also and are not limited, in any way to the models used therein. Furthermore, a plurality of mathematical models can be used as curve fitting models for the data obtained using the SCTV viscometers, as disclosed in application Ser. No. 09/908,374 and thus the model used in that disclosure is by way of example only also and is not limited, in any way to the model used therein. As a result, the particular details of all of these disclosures is not repeated here but are all incorporated by reference herein.

In view of all of the above, these DPD viscometers operate in accordance with the method of the present invention 2000:

In step 2001, a column of fluid is formed in a substantially upright lumen having a first end and a second end.

In step 2002, a start point is established of the column of fluid above a horizontal reference position (e.g., DATUM or “ref”).

In step 2003, the first and second ends of the lumen are then exposed to atmospheric pressure to subject the column of fluid to a continuously decreasing pressure differential that causes the column of fluid to move away from the start point towards the second end through a plurality of shear rates.

In step 2004, as the column of fluid moves, the movement is restricted by its, or a portion of the column's, passage through the flow restrictor, e.g., a capillary tube, having some known dimensions, e.g., diameter and length.

In step 2005, as the column of fluid is moving, this movement is monitored through the plurality of shear rates in order to generate data related to the movement (e.g., changing column height, changing mass/weight, changing volume, changing position, time of flight, etc.).

In step 2006, the fluid viscosity is calculated using the data and the known dimensions of the flow restrictor.

Thus, the above represent exemplary DPD viscometers that can be used to determine the viscosity of a fluid over a plurality of shear rates, including biological fluids such as blood.

Without further elaboration, the foregoing will so fully illustrate our invention that others may, by applying current or future knowledge, readily adopt the same for use under various conditions of service.

We claim:

1. An apparatus for determining the viscosity of a fluid over plural shear rates using a continuously decreasing pressure differential, said apparatus comprising:
a lumen for supporting a column of fluid therein and wherein said column of fluid has a start point defined above a horizontal reference position, said lumen comprising:
a first end and a second end;
a flow restrictor for restricting the movement of said column of fluid and located between said first and second ends, said flow restrictor comprising some known dimensions; and
wherein, after said start point has been defined, said first and second ends are exposed to atmospheric pressure to subject said column of fluid to a continuously decreasing pressure differential that causes said column of fluid to move away from said start point towards said second end through a plurality of shear rates;
a sensor for monitoring the movement of said column of fluid in said lumen for generating data related to the movement; and
a processor for using said data and said some known dimensions to calculate the viscosity of the fluid.

2. The apparatus of claim 1 wherein said lumen comprises a U-shaped structure and wherein said flow restrictor comprises a capillary tube of known diameter and length and which forms a part of said U-shaped structure.

3. The apparatus of claim 2 wherein the column of fluid has a forward edge and a trailing edge and wherein said sensor comprises an optical detector to monitor the movement of said forward edge.

4. The apparatus of claim 3 further comprising a second optical sensor which monitors the movement of said trailing edge.

5. The apparatus of claim 3 further comprising a second optical sensor which detects the trailing edge at said starting point.

6. A method for determining the viscosity of a fluid over plural shear rates using a continuously decreasing pressure differential, said method comprising the steps of:
forming a column of the fluid in a substantially upright lumen having a first end and a second end;
establishing a start point of said column of fluid above a horizontal reference position;
exposing said first end and said second end to atmospheric pressure to subject said column of fluid to a continuously decreasing pressure differential that causes said column of fluid to move away from said start point towards said second end through a plurality of shear rates;
restricting the movement of said column of fluid by passing at least a portion of said column of fluid through a flow restrictor having some known dimensions;
monitoring the movement of said column of fluid through said plurality of shear rates to generate data related to said movement; and
calculating the viscosity of the fluid using said data and said some known dimensions.

7. The method of claim 6 wherein said step of restricting the movement of said column comprises having at least a portion of the column of fluid pass through a capillary tube having a known diameter and length.

8. The method of claim 6 wherein the column of fluid has a forward edge and a trailing edge and wherein said step of monitoring the movement of said column of fluid comprises using an optical detector to monitor the movement of said forward edge.

9. The method of claim 8 wherein said step of monitoring the movement of said column also comprises monitoring the movement of said trailing edge.

10. The method of claim 8 wherein said step of monitoring the movement of said column also comprises detecting the trailing edge at said starting point.

11. The method of claim 6 wherein the second end is positioned over a fluid collector positioned on a mass detector and wherein said step of monitoring the movement of said column of fluid comprises detecting the increasing weight of said fluid collector.

12. A method for determining the viscosity of a fluid over plural shear rates using a continuously decreasing pressure differential, said method comprising the steps of:
forming a column of the fluid in a substantially upright lumen having a first end and a second end, said lumen having some known dimensions;
establishing a start point of said column of fluid above a horizontal reference position;
exposing said first end and said second end to atmospheric pressure to subject said column of fluid to a continuously decreasing pressure differential that causes said column of fluid to move away from said start point towards said second end through a plurality of shear rates, said substantially upright lumen restricting the movement of said column of fluid as it moves;
monitoring the movement of said column of fluid through said plurality of shear rates to generate data related to said movement; and
calculating the viscosity of the fluid using said data and said some known dimensions.

13. The method of claim 12 wherein the column of fluid has a forward edge and a trailing edge and wherein said step of monitoring the movement of said column of fluid comprises using an optical detector to monitor the movement of said forward edge.

14. The method of claim 13 wherein said step of monitoring the movement of said column also comprises monitoring the movement of said trailing edge.

15. The method of claim 13 wherein said step of monitoring the movement of said column also comprises detecting the trailing edge at said starting point.
